(19) United States
(12) Patent Application Publication
(10) Pub. No.: US 2003/0163190 A1
(43) Pub. Date: Aug. 28, 2003

(54) High temperature stent delivery system

(75) Inventors: Antoine LaFont, Paris (FR); Eric J. Simso, Excelsior, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: SCIMED LIFE SYSTEMS, INC.

(21) Appl. No.: 10/084,294

(22) Filed: Feb. 25, 2002

(51) Int. Cl. A61F 2/06

(52) U.S. Cl. 623/1.11; 600/101

(57) ABSTRACT

A method and apparatus for the treatment of a bodily vessel. The apparatus comprising a catheter having a means for heating a desired location of the bodily vessel. The method comprising delivering a distal portion of the catheter to the desired location in a bodily vessel and activating the means for heating in order to heat the vessel at the desired location. A stent may be delivered and implanted to the bodily vessel at the desired location.

### BACKGROUND OF THE INVENTION

Narrowing or blockage of bodily lumens or vessels such as: coronary and peripheral veins or arteries, the gastrointestinal tract, biliary ducts, esophagus, urethra, tracheal and bronchial ducts, may occur for a variety of reasons. For example, a stenosis of an artery is a constriction or narrowing of the artery that may result from the buildup up of cholesterol, fat or other substances. Stenoses of coronary arteries (coronary artery disease) for example, can diminish the flow of blood to the heart leading to heart damage and, possibly, death. Currently coronary artery disease is one of the leading causes of death worldwide. A number of methods have been developed for treating coronary arteries and other bodily vessels or lumens which have become blocked. These methods include stenting, percutaneous transluminal coronary angioplasty (PTCA), coronary artery bypass grafting (CABG) and the use of intracoronary drugs and/or radiation.

Coronary stenting is an established treatment for the majority of patients with symptomatic coronary artery disease. In 2001, it is estimated that roughly 2 million stents will be implanted worldwide. The main benefit of stenting compared with PTCA consists of the reduction of re-narrowing or restenosis. The mechanism of this reduction is largely a function of achieving a bigger initial lumen with stenting and a corresponding reduction in the acute recoil of the vessel. Despite this initial gain, stenting is usually accompanied by an increased late loss, which is generally a function of excessive growth of scar tissue (neointima) growth. While the initial gain is usually more than sufficient to compensate for the greater loss in lumen area (neointimal growth) that occurs with stenting, some percentage of stented arteries require re-treatment due to excessive neointimal growth. This condition is referred to as restenosis.

Recent clinical trials have suggested that restenosis occurs in a fraction of stented patients. Estimates of clinical restenosis rates range from as low as roughly 15% of patients to as high as 30% to 40% for certain devices and vessels or lesions. A number of treatments have been used to treat in-stent restenosis, but most are marginally effective, and restenosis remains difficult to treat in patients in whom it occurs. Restenosis typically occurs within less than six months of initial treatment of the vessel but may manifest itself many months to years later.

A number of different techniques have been devised to reduce the likelihood of restenosis in stented regions of a vessel. These techniques include treating the patient with anticoagulant and antiplatelet drugs and smooth muscle cell inhibitors. Direct delivery of drugs to the affected vessel is addressed in a number of patents including U.S. Pat. No. 5,861,168 which discloses the use of a stent bearing a nitric oxide precursor agent and U.S. Pat. No. 5,800,507 which discloses the use of a stent bearing fibrin. Other methods of treatment include delivery of radioactive substances to the affected region of the body as is disclosed in U.S. Pat. Nos. 5,871,437, 5,059,166 and 6,099,455. The use of ultrasonic energy in reducing the likelihood of restenosis is disclosed U.S. Pat. No. 5,836,896.

A method that has been investigated to prevent restenosis in combination with balloon angioplasty is the delivery of heat during or immediately after PTCA. This method of treatment and an associated device has been disclosed in U.S. Pat. No. 6,190,355. In a multi-center clinical study, such a device and method did show reduced neointimal formation when used in combination with PTCA. Unfortunately, the device showed no improvement in clinical restenosis rates, primarily due to excessive late loss caused by increased vessel recoil (scar formation). This approach and corresponding device has not been described or studied in conjunction with the placement of a vascular stent.

All U.S. patents and applications and all other published documents mentioned anywhere in this application are incorporated herein by reference in their entirety.

Without limiting the scope of the invention in any way, the invention is briefly summarized in some of its aspects below. Additional details of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF THE INVENTION

In one embodiment, the invention is directed to a method of treating a bodily lumen or vessel comprising the steps of delivering a catheter, and delivering heat to a desired location of the lumen by heating contrast agent in a portion of the catheter underlying the desired location. In accordance with the invention, a stent may also be delivered prior to the use of this catheter, or a stent may be delivered via the described catheter. The stent is preferably a balloon expandable or self expandable metallic stent, but may also be a polymeric or biodegradable stent. Metallic stents may be stainless steel, nitinol or any number of metallic alloys. The stent and stented region of the vessel may be heated by the catheter before, during or after the stent is implanted. The contrast agent would preferably be enclosed within a conventional PTCA balloon and heated by a number of methods including RF energy, injection of pre-heated contrast through a balloon inflation lumen, ultrasonic heating of the balloon in-situ, or magnetic heating.

In the preferred embodiment, the catheter is a conventional PTCA catheter, commonly used in PTCA and stent delivery. This catheter has an internal electrode at the balloon or distal end, which is connected at the hub, or proximal end to a device, which generates RF energy. The catheter is inflated with contrast agent as is normally done in PTCA, and the heating energy is applied, causing heating at the distal end of the catheter. The heat range is from 40 degrees C. to 99 degrees C. for time periods from 1 second to 5 minutes.

In another embodiment, the invention is directed to a stent delivery apparatus comprising a catheter, a stent and an ultrasonic transducer element for generating ultrasonic waves. Desirably, the stent is disposed at the distal region of the catheter. Also desirably, the ultrasonic transducer element is disposed at the distal region of the catheter. Optionally, a plurality of ultrasonic transducer elements may be provided.

In another embodiment, the invention is directed to a stent delivery apparatus comprising a catheter having a distal region, a stent and a magnetic medium which may be inductively heated via the application of electromagnetic energy thereto. The magnetic medium may form a portion of the stent or a portion of the catheter.

Additional details and/or embodiments of the invention are discussed below.

### BRIEF DESCRIPTION OF THE FIGURES

A detailed description of the invention is hereafter described with specific reference being made to the drawings in which:

A detailed description of the invention is hereafter described with specific reference being made to the drawings in which:

FIG. 1 is a side view of an embodiment of the invention;

FIG. 2 is a side view of an alternative embodiment of the invention;

FIG. 3 is a side view of an alternative embodiment of the invention;

FIG. 4 is a cross-sectional view of the embodiment shown in FIG. 3 taken along the Y-axis, indicated in FIG. 3.

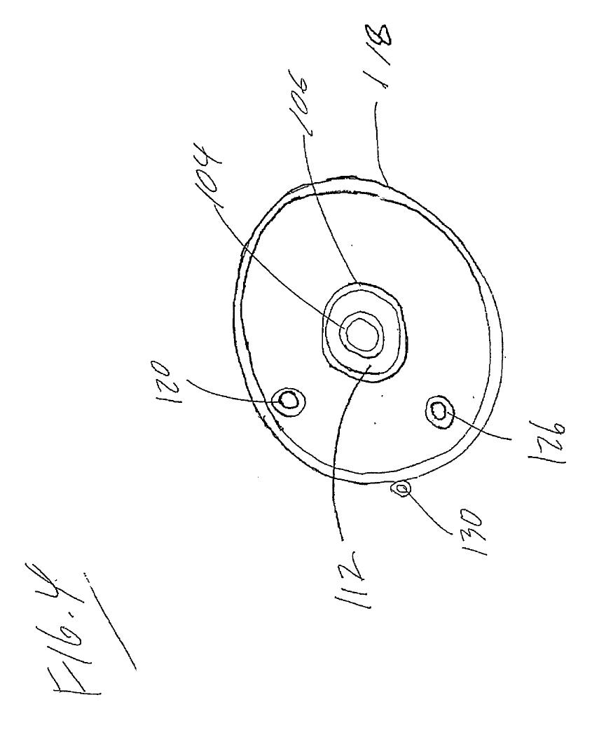

FIG. 5 is a side view of an alternative embodiment of the invention;

FIG. 6 is a schematic view of the embodiment of the invention shown in FIG. 3.

FIG. 7 is a side view of an alternative embodiment of the invention; and

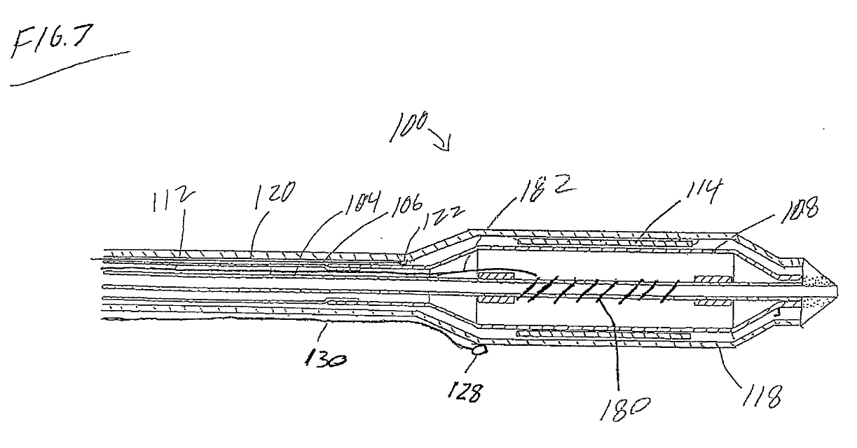

FIG. 8 is a schematic view of the embodiment of the invention shown in FIG. 7.

### DETAILED DESCRIPTION OF THE INVENTION

While this invention may be embodied in many different forms, there are shown in the drawings and described in detail herein specific embodiments of the invention. The present disclosure is an exemplification of the principles of the invention and is not intended to limit the invention to the particular embodiments illustrated.

For the purposes of this disclosure, unless otherwise indicated, identical reference numerals used in different figures refer to the same component.

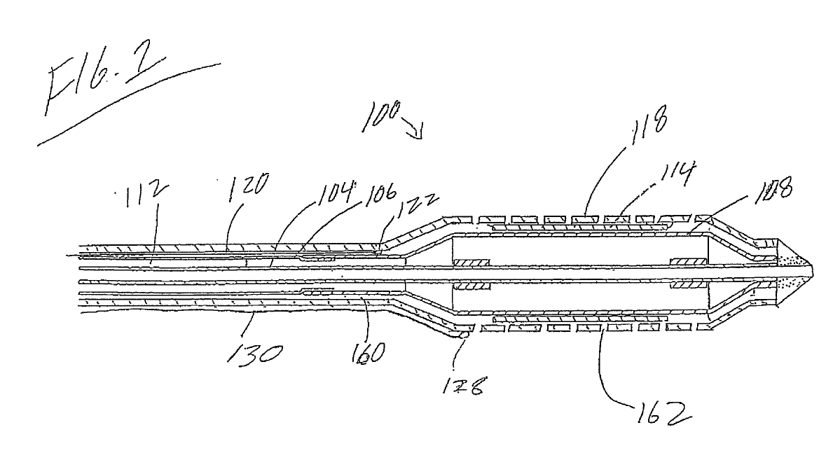

In one embodiment, the invention is directed to a catheter such as that shown generally at 100 in FIG. 1. Catheter 100 may be any type of catheter. For example, catheter 100 may be a stent delivery catheter and/or a perfusion catheter. The catheter may also be suitable for drug delivery applications as discussed below. Catheter 100 includes an inner member 104 about which stent 114 is disposed and an outer member 106. Inner member 104 may be of any suitable construction. For example, inner member 104 may in the form of a tube or may be solid. Outer member 106 is disposed about inner member 104. Outer member 106 may be in the form of a tube. Outer member 106 and inner member 104 may also be in the form of a dual lumen tube. In the case of a balloon expandable stent, the balloon 108 is disposed about inner member 104, desirably at the distal end of the outer member 106. Stent 114 is disposed about balloon 108. In the case of a self expanding stent, the retractable sheath 118 will be present and is described in detail later.

Stent 114 may be mechanically expandable, self-expanding or a hybrid of the two. Where stent 114 is mechanically expandable, the balloon as shown in FIG. 1, is provided to expand the stent. Any suitable stent may be used including stents which are tubular in the unexpanded state and stents which are in the form of rolled sheets in the unexpanded state. Desirably, the stent will be made of a metal such as stainless steel, MP35N, tantalum, platinum, gold, titanium, Elgiloy and Phynox or any alloys thereof. Other suitable metals include shape memory metals such as nitinol.

Retractable sheath 118 is disposed about stent 114 in the case of a self expanding stent. Desirably, such a self expanding stent will be made of a suitable shape memory metals such as nitinol, tantalum, Elgiloy and Phynox or any alloys thereof. Other self expanding stents such as wire braid designs are also part of the invention. Retractable sheath 118 may be retracted using any suitable retraction mechanism. An example of a suitable retraction mechanism comprising pull wire 120 and pull collar 122 is shown in FIG. 1. Pull wire 120 extends proximally from pull collar 122. Pull collar 122, in turn, extends from retractable sheath 118.

Catheter 100 further comprises a means of heating the contrast agent within the balloon or within the sheath. The stent and/or vessel may be heated via the use of RF energy using a catheter such as that disclosed in U.S. Pat. No. 6,190,355, modified for stent delivery. The preferred method of heating is by using RF energy transmitted to the contrast agent via a transducer 180 placed under the balloon or sheath. The transducer is connected to an external RF energy source through wires or other electromagnetic connections 182 to the hub or proximal end of the catheter and from there to as source of RF energy 184.

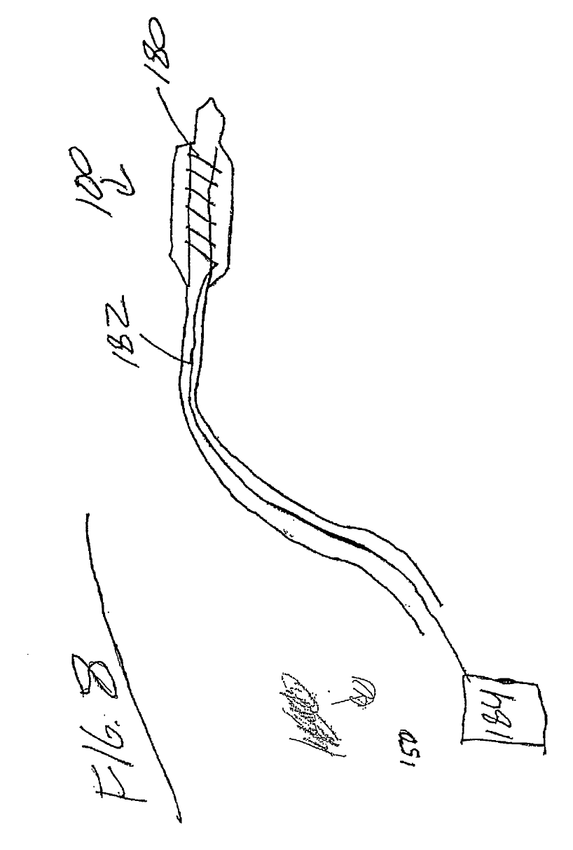

Other methods of heating the surrounding environment include embodiments of the invention wherein the catheter 100 includes one or more electrically resistive elements 180, which are incorporated into and/or disposed about a portion of the catheter 100. While the present invention may include electrically resistive element 180 in any portion of the catheter 100, in the embodiment shown electrically resistive element 180 is disposed about the inner member 104. Electrically resistive element 180 has a conductive element 182, which extends proximally to an electric power source 184 such as shown in FIG. 8. Desirably, electrically resistive element 180 is in the form of one or more wires and conductive element 182 is in the form of one or more wires.

When a predetermined electric current is delivered to the electrically resistive element 180, the electrically resistive element may be made to heat up thereby, radiantly and conductively, increasing the temperature of the surrounding vessel. The precise temperature may be manipulated by lowering the amount of electricity supplied to the electrically resistive element 180. The temperature may be monitored via temperature probe 128 in the manner previously described.

The inventive catheters may be provided in rapid exchange form, in over-the-wire form, in hybrid form, in fixed-wire embodiments as well as in other forms.

In addition to being directed to the specific combinations of features claimed below, the invention is also directed to embodiments having other combinations of the dependent features claimed below and other combinations of the features described above.

The inventive catheters of the present invention may be further provided with a temperature probe 128 and connecting wire 130 or other electrically conducting member to a thermometric device (not shown) to monitor the temperature of the desired bodily location. The temperature of the contrast agent may be adjusted, as necessary to maintain the desired temperature of vessel at the desired location.

In use, catheter 100 may be used to deliver stent 114 to a desired bodily location. Either immediately before, during or after implantation of the stent, the surrounding vessel and/or stent may be heated by delivering a heated contrast agent to the desired bodily location. In the case of a self-expanding stent where the stent is delivered first, retractable sheath 118 may be retracted, and the stent is allowed to self expand. In the case of a balloon expandable stent, an inflation fluid may be delivered to balloon 108 to expand balloon 108 and stent 114.

Any suitable contrast agent may be used. Where the implantation of the stent is monitored via fluoroscopy, a fluoroscopy contrast agent, such as an iodine based agent or others, may be used. Contrast agents are well known in the field of fluoroscopy. Where the implantation of the stent is monitored via magnetic resonance imaging (MRI), suitable MRI contrast agents may be used. Examples of suitable MRI contrast agents include gadolinium based compounds such as gadolinium-DTPA, ferrite particles and other iron based species.

Desirably, the contrast agent will be heated to a temperature ranging from about 37° C.-99° C. for a period of time ranging from 1 second to 5 minutes.

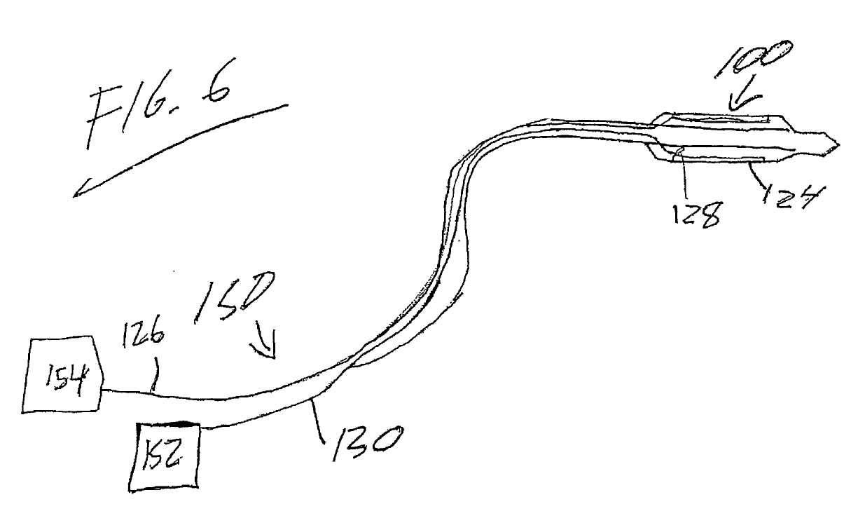

Suitably, transducer element 124 is made of a piezoelectric material and a protective material for the purposes of mechanical and chemical isolation as well as for electric insulation. One or more electrically conducting members 126, desirably in the form of wire, are in electrical communication at the proximal end 150 of the catheter 100 with transducer element 124 and a controller 154, as shown in FIG. 6. Any suitable control device may be used to control the transducer element.

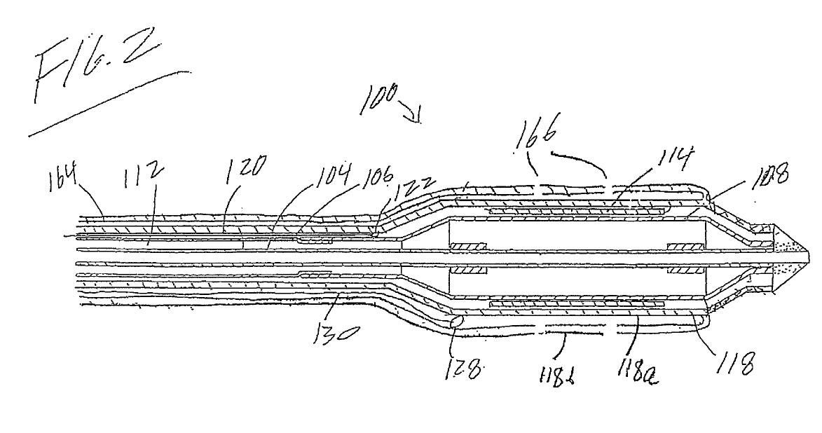

As shown in FIG. 1 it may be desirable to provide catheter 100 with a temperature probe 128 and conducting element, desirably in the form of a connecting wire 130, to monitor the temperature of the desired bodily location. As shown in FIG. 2 the connecting wire 130 may be connected at the proximal end 150 of the catheter 100 to a thermetric device within 184 for monitoring the temperature detected by probe 128. Connecting wire 130 may run exterior to the catheter, interior to the catheter or be exterior to the catheter for part of the length of the catheter and interior for another part of the length of the catheter.

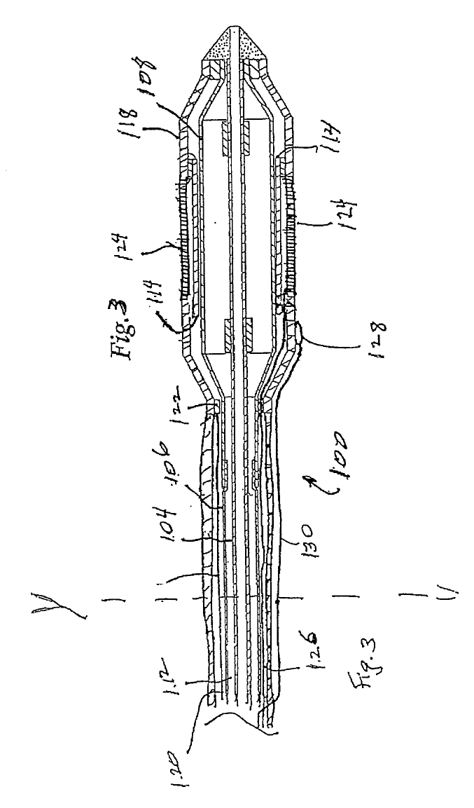

As shown in FIG. 3, the catheter shown in FIG. 1 is illustrated in cross-section, along the Y-axis as labeled in FIG. 3. Connecting wire 130 may be positioned external of the sheath 118. However, if desired the wire may be positioned anywhere within the catheter 100. Similarly, wires 120 and 126 may be positioned external to the sheath 118 or elsewhere. For example, wires 120 and 126 could be positioned between the inner member 104 and the outer member 106.

In use, catheter 100 may be used to deliver stent 114 to a desired bodily location. Either immediately before, during or after implantation of the stent, the surrounding vessel and/or stent may be heated by delivering heating energy with transducer 124. Where the stent is delivered first, retractable sheath 118 may be retracted. In the case of a self-expanding stent, the stent is allowed to self expand. In the case of a balloon expandable stent, an inflation fluid may be delivered to balloon 108 to expand balloon 108 and stent 114. In either case if the heat energy is delivered during or after stent deployment, the heated contrast will be contained within a balloon. If the energy is delivered prior to stent deployment the energy may be delivered inside the balloon, outside the inner lumen or inside the sheath. Energy delivered may be in the form of selected radio frequencies (RF), ultrasonic, or other types of energy.

It is also within the scope of the invention for the transducer element to be longitudinally displaced from the stent, either distal to or proximal to the stent. In such an embodiment, it may be necessary for the catheter to be repositioned prior to or subsequent to deployment of the stent to deliver the ultrasonic energy to the desired location in the vessel.

Moreover, the transducer need not be provided on the inner lumen. Other locations for the transducer element are also within the scope of the invention including locations on the retractable sheath and elsewhere. For example, the stent may be disposed about the transducer element.

Once the stent 114 has been delivered, the surrounding vessel may be heated, via the delivery of ultrasonic energy, to a temperature ranging from about 37° C.-99° C.

In another embodiment of the invention, the invention is directed to a stent delivery apparatus such as shown in FIGS. 1-3 wherein the catheter may include a magnetic medium which may be inductively heated via the application of radio frequency electromagnetic energy thereto. Any of the types of stents disclosed above may be employed in the invention.

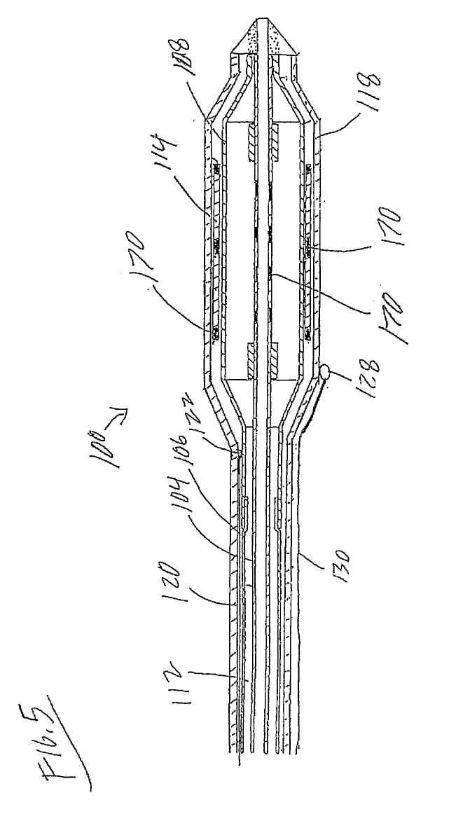

As is shown in FIG. 5, the magnetic medium 170 may form a portion of the stent 114, a portion of the catheter 100 or any combination thereof. Where the magnetic medium 170 forms a portion of the stent 114, any suitable stent delivery system known in the art may be used. Suitable magnetic materials for such a stent include magnetic ferrite or ‘ferrite’ which is a substance consisting of mixed oxides of iron and one or more other metals. Other suitable materials include those disclosed in U.S. Pat. No. 5,441,746. Known stent materials such as nitinol and stainless steel may also be rendered sufficiently magnetic by subjecting the stent material to a sufficient electric and/or magnetic field. Further details concerning magnetic stents may be found in the copending, commonly assigned U.S. application Ser. No. 09/808854, filed Mar. 15, 2001 and entitled “Magnetic Stent”.

Where the magnetic medium forms a portion of the catheter 100, magnetic particles such as those disclosed above may be incorporated into one or more portions of a catheter. For example, magnetic particles may be incorporated into a portion of an inner tube 104 of a stent delivery system in the vicinity of a stent, such as is shown in FIG. 5, or elsewhere. Magnetic particles may also be incorporated into the retractable sheath portion 118 of a catheter 100 such as is illustrated in FIG. 1.

In use, where a magnetic medium is employed, the stent may be delivered to a desired location in a bodily vessel and prior to, during or subsequent to deployment of the stent, a strong electromagnetic field may be directed toward the desired bodily location. Such a field may typically be generated in a magnetic resonance imager. The field is applied for a sufficient period of time to cause the desired bodily location to heat to a desired temperature. Suitably, the temperature may be monitored via a temperature probe 128 such as has been previously described.

In the present embodiment, the magnetic medium 170 may be inductively heated before, during or after the stent is implanted. As indicated above, the surrounding vessel may be heated, to a temperature ranging from about 37° C.-99° C.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A method of treating a bodily vessel comprising the steps of:
inserting a catheter having a distal portion into a body vessel;
advancing the distal portion to a desired location in a bodily vessel; and
delivering heat to the location by a variety of means.

2. The method of claim 1 wherein at least a portion of the distal end of the catheter includes a stent disposed thereabout.

3. The method of claim 2 further comprising the step of delivering the stent.

4. The method of claim 3 wherein the heated contrast agent is delivered to the distal end after the stent is delivered.

5. The method of claim 3 wherein the heated contrast agent is delivered to the distal end as the stent is delivered.

6. A method of treating a bodily vessel comprising the steps of: advancing a stent delivery catheter comprising a stent to a desired location in a bodily vessel; implanting the stent in the bodily vessel at the desired location; and heating the bodily vessel at the desired location.

7. The method of claim 6 wherein the bodily vessel is inductively heated by directing energy to a portion of the catheter.

8. The method of claim 6 wherein the bodily vessel is inductively heated by directing energy to at least a portion of the stent.

9. The method of claim 6 wherein the bodily vessel is inductively heated by directing an RF electromagnetic field to the desired location.

10. The method of claim 6 wherein the bodily vessel is inductively heated at the desired location after the stent is implanted.

11. The method of claim 6 wherein the bodily vessel is inductively heated at the desired location as the stent is implanted.

12. The method of claim 6 wherein the bodily vessel is inductively heated at the desired location immediately before the stent is implanted.

13. A method of treating a bodily vessel comprising the steps of:
delivering a stent to a desired location in a bodily vessel;
implanting the stent in the bodily vessel at the desired location; and
heating the stent at the desired location.

14. The method of claim 13 wherein the stent is ultrasonically heated.

15. The method of claim 14 wherein the bodily vessel is ultrasonically heated at the desired location after the stent is implanted.

16. The method of claim 14 wherein the bodily vessel is ultrasonically heated at the desired location as the stent is implanted.

17. The method of claim 10 wherein the bodily vessel is ultrasonically heated at the desired location immediately before the stent is implanted.

18. A stent delivery apparatus comprising:
a catheter having a distal region and an ultrasonic transducer element positioned within the distal region, the ultrasonic transducer element constructed and arranged to generate ultrasonic waves.

19. The stent delivery apparatus of claim 18 further comprising a stent, the stent being disposed about at least a portion of the distal region of the catheter.

20. A stent delivery apparatus comprising:
a catheter having a distal region;
a stent, the stent disposed about at least a portion of the distal region;
a resistive metal element positioned in proximity with the stent; and
a source of electricity in electrical communication with the metal element.

21. A stent delivery apparatus comprising:
a catheter having a distal region;
a stent, the stent disposed about at least a portion of the distal region;
a magnetic wave absorbing moiety in the vicinity of the stent; and
a source of radio frequency waves absorbable by the magnetic wave absorbing moiety.

22. A method of treating a bodily vessel comprising the steps of:
inserting a catheter into a bodily vessel;
advancing a distal portion of the catheter to a desired location within the bodily vessel;
delivering a magnetic medium to the distal portion of the catheter; and
inductively heating the magnetic medium.

23. The method of claim 22 wherein a stent is at least partially disposed about the distal portion of the catheter.

24. The method of claim 23 further comprising the steps of delivering the stent to the desired location.

25. The method of claim 24 wherein the magnetic medium is inductively heated as the stent is implanted.

26. The method of claim 24 wherein the magnetic medium is inductively heated after the stent is implanted.

27. The method of claim 24 wherein the magnetic medium is inductively heated before the stent is implanted.

28. The method of claim 24 wherein the magnetic medium forms a portion of the catheter.

29. The method of claim 22 wherein the magnetic medium is inductively heated by application of radio frequency electromagnetic energy thereto.
